(19) United States
(12) Patent Application Publication
(10) Pub. No.: US 2017/0274174 A1
(43) Pub. Date: Sep. 28, 2017

(54) SYSTEM AND METHOD FOR ADMINISTERING, MONITORING AND CONTROLLING BIOMIMETIC SLEEP

(71) Applicant: THE GENERAL HOSPITAL CORPORATION, Boston, MA (US)

(72) Inventors: Patrick L. Purdon, Somerville, MA (US); Emery N. Brown, Brookline, MA (US); Christ J. Van Dort, Cambridge, MA (US); Olewaeseun Akeju, Dorchester, MA (US)

(73) Assignee: The General Hospital Corporation, Boston, MA (US)

(21) Appl. No.: 15/505,219

(22) PCT Filed: Aug. 24, 2015
(86) PCT No.: PCT/US2015/046602

§ 371 Date: Feb. 20, 2017

Related U.S. Application Data
(60) Provisional application No. 62/040,831, filed on Aug. 22, 2014.

(51) Int. Cl. A61M 21/02; A61B 5/0476; A61B 5/04; A61B 5/00

(52) U.S. Cl. CPC A61M 21/02; A61B 5/4812; A61B 5/0476; A61B 5/4839; A61B 5/04012; A61M 2230/10; A61M 2230/18; A61M 2021/0077

(57) ABSTRACT

Systems and methods for inducing natural sleep in a subject is provided. In some aspects, the method includes inducing a first sleep state; monitoring a first plurality of characteristics of a brain of the subject to verify the subject is in the first sleep state; inducing a second sleep state after the subject has been in the first sleep state for a predetermined amount of time; and monitoring a second plurality of characteristics of the brain of the subject to verify the subject is in the second sleep state.

### CROSS-REFERENCE TO RELATED APPLICATIONS

This application claims the benefit of U.S. Provisional Patent Application Ser. No. 62/040,831 filed on Aug. 22, 2014 and entitled “SYSTEM AND METHOD FOR ADMINISTERING, MONITORING AND CONTROLLING BIOMIMETIC SLEEP.”

### STATEMENT REGARDING FEDERALLY SPONSORED RESEARCH

This invention was made with government support under DP1-OD003646, R01-GM104949, and DP2-OD006454 awarded by National Institutes of Health. The government has certain rights in the invention.

### BACKGROUND

The field of the invention is related to systems and methods for monitoring and controlling biomimetic sleep in a subject. More particularly, the invention is directed to systems and methods for monitoring and controlling biomimetic sleep using pharmacological, electrical and optogenetic techniques.

Sleep is a neural process consisting of multiple local and spatiotemporally- evolving factors, and direct observation of brain activity has been the primary means of characterizing sleep. Since the first recordings of brain activity using electroencephalogram (“EEG”) data, scientists have sought to characterize the complex recurring patterns associated with sleep. Primarily, these patterns have been organized through the process of sleep staging, which is a rule-based categorization of sleep performed using visual inspection of the EEG time-series in non-overlapping epochs.

In the 1920s, Hans Berger, inventor of the EEG, first noted the difference between the sleeping and wake EEG, and observed the occipital oscillation in alpha (8-12 Hz). In the 1930s, Loomis, Harvey and Hobart incorporated the presence of spindles in the sigma band (12-15 Hz), and proposed a 5-stage categorization of sleep. In the 1950s, rapid eye movement (“REM”) sleep was discovered by Aserinsky and Kleitman. These discoveries paved the way for the Rechtschaffen and Kales (“R&K”) system in 1968 of categorizing sleep stages. In this system, EEG recordings were visually scored over 30-second epochs, differentiating the awake stage from a rapid eye movement (“REM”) stage and four non-rapid eye movement (“NREM”) stages of sleep. Almost 50 years later, R&K scoring remains the clinical standard for sleep medicine and sleep research, with the minor modification that reduces NREM sleep to three stages, namely N1-N3. This approach provides a manageable abstraction and discretization of the EEG activity observed during the sleep, allowing crude identification of major sleep landmarks and instantaneous transitions during sleep.

During natural sleep, humans generally switch between REM and NREM states. In particular, REM sleep is characterized by rapid eye movements, emotion-laden dreaming, irregularities of respiration and heart rate, genital erection, airway and skeletal muscle atonia, and active, high-frequency EEG oscillations. NREM sleep is generally characterized by high amplitude, low frequency EEG oscillations, decreased muscle tone, body temperature and heart rate. There can be up to three stages of NREM sleep in natural human sleep. Therefore, natural sleep can be defined as a programmed set of oscillations between REM sleep and three stages of NREM sleep. This oscillatory dynamic is critical for achieving the restorative benefits of natural sleep. Methods and systems for determining these sleep states can be found in PCT Application No. US 2015/028046, entitled “System and Method for Spectral Characterization of Sleep,” filed Apr. 28, 2015, and which is hereby incorporated by reference in its entirety.

Natural sleep is critical for maintaining cardiovascular, immune and cognitive functions. However, sleep disorders such as sleep apnea, insomnia and narcolepsy affect some 60 million persons in the United States alone. Additionally, psychiatric diseases, such as depression, and neurological diseases such as Parkinson's are associated with significant sleep disruption. The principal sleep medications, benzodiazepines, eszopiclone, and zolpidem, are among the most widely sold pharmaceuticals used to treat insomnia or other sleep related issues. These drugs generally target inhibitory GABA receptors throughout the brain. The drug formulations are designed for immediate release or timed release based on whether the insomnia is due to difficulty falling asleep or difficulty staying asleep, respectively. However, these sleep medications do not work to selectively activate the brain circuits in a precisely timed manner to drive the typical 90 minute NREM-REM cycling of natural sleep, required to obtain the proper restorative benefits. Instead, the above medications work to globally inhibit brain activity, and, at best, produce only sedation. Further, NREM sleep can be a factor in improving memory function, and REM sleep can be instrumental in memory consolidation. These benefits can be lost when sedation inducing sleep aids are administered.

Therefore, given the above, there is a need for systems and methods for use in controlling sleep. In particular, the ability to monitor a subjects sleep state and then control the sleep of the subject using pharmacological, optogenetics, and/or electronics to produce biomimetic sleep.

### SUMMARY

The present invention overcomes the drawbacks of aforementioned technologies by providing a system and method for controlling sleep of a subject by inducing sleep states, such as NREM and REM sleep states.

In accordance with one aspect of the disclosure, a method for inducing natural sleep in a subject is provided. The method includes inducing a first sleep state; monitoring a first plurality of characteristics of a brain of the subject to verify the subject is in the first sleep state; inducing a second sleep state after the subject has been in the first sleep state for a pre-determined amount of time; and monitoring a second plurality of characteristics of the brain of the subject to verify the subject is in the second sleep state.

In accordance with another aspect of the disclosure, a system for controlling sleep states of a subject is provided. The system includes a sensor, wherein the sensor assembly is capable of measuring brain activity. A sleep controller, the sleep controller in communication with the sensor assembly and including an input, an output and a processor, wherein the sleep controller analyzes the measured brain activity. And, an external control module, the external control module in electronic communication with the sleep controller and capable of inducing a sleep state in a subject.

In accordance with a further aspect of the disclosure, a method for inducing biomimetic sleep in a subject is provided. The method includes inducing a first sleep state; monitoring a first plurality of characteristics of a brain of the subject to verify the subject is in the first sleep state; inducing a second sleep state after the subject has been in the first sleep state for a pre-determined amount of time; monitoring a second plurality of characteristics of the brain of the subject to verify the subject is in the second sleep state; and transitioning between the first sleep state and the second sleep state for a pre- determined number of transitions, wherein there is a pre-determined time delay between inducing the second sleep state and the first sleep state.

The foregoing and other aspects and advantages of the invention will appear from the following description. In the description, reference is made to the accompanying drawings that form a part hereof, and in which there is shown by way of illustration of preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

FIG. 1 is cross-sectional image of a typical human brain.

FIG. 2A is a graphical example illustrating spectral dynamics during NREM progression into slow wave sleep in humans.

FIG. 2B illustrates waveform EEG data corresponding to different time points for the spectrogram of FIG. 2A.

FIG. 3A is a graphical example illustrating spectral dynamics during NREM stage 2 sleep induced with low-dose administration of dexmedetomidine

FIG. 3B illustrates waveform EEG data corresponding to different time points for a specific time point of the spectrogram of FIG. 3A.

FIG. 4A is a graphical example illustrating spectral dynamics during NREM stage 3 sleep induced with high-dose administration of dexmedetomidine.

FIG. 4B illustrates waveform EEG data corresponding to different time points for a specific time point of the spectrogram of FIG. 4A.

FIG. 5 is a flow chart illustrating a process for inducing natural sleep.

FIG. 6 is a schematic view of a Brain-Machine-Interface.

FIG. 7 is an example sensor assembly for acquiring electroencephalogram (“EEG”) data, in accordance with aspects of the present disclosure.

FIG. 8 shows non-limiting sleep monitor example embodiments, in accordance with aspects of the present disclosure.

FIG. 9 shows additional non-limiting sleep controller example embodiments, in accordance with aspects of the present disclosure.

FIG. 10 illustrates a process for inducing natural sleep using a Brain-Machine-Interface in accordance with aspects of the present disclosure.

### DETAILED DESCRIPTION

Natural human sleep generally comprises two separate stages. Non-rapid eye movement (“NREM”) sleep, and rapid eye movement (“REM”) sleep. Throughout the course of the night, humans normally transition between NREM and REM sleep several times, with a general period of ninety minutes. Different sleep stages exhibit different detectable signatures, such as delta power, K-complexes, and sleep spindle activity, indicative of various brain states associated with sleep. Therefore, it is a discovery of the present disclosure that sleep patterns could be controlled, for instance, using electroencephalogram (“EEG”) and other measurements, in a manner that can achieve natural sleep using systems and methods described herein.

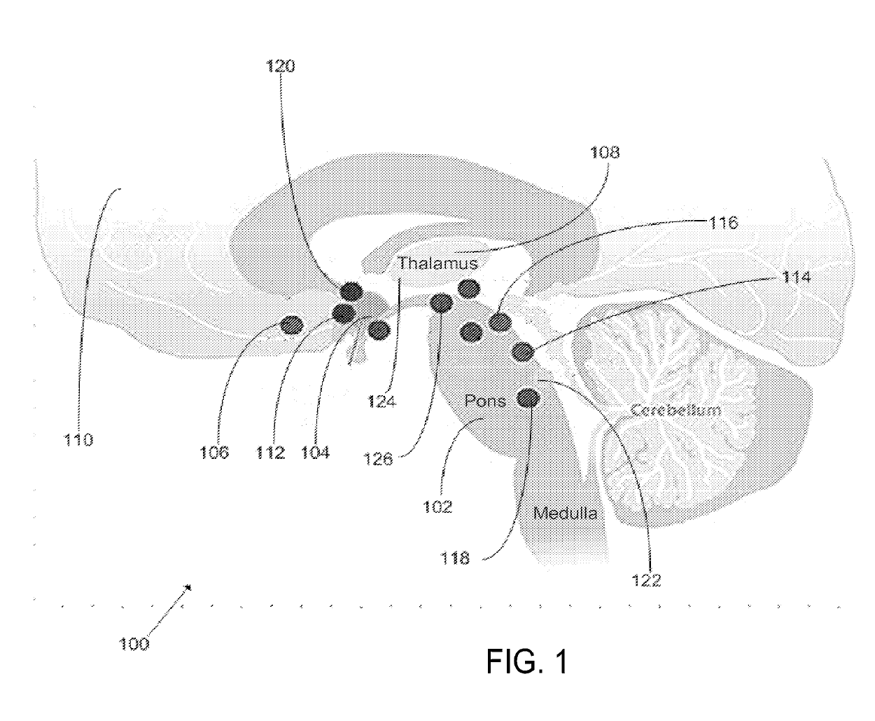

Turning to FIG. 1. some of the principal arousal centers of the human brain 100 that lie in the midbrain are shown, which can include the pons 102, hypothalamus 104 and basal forebrain 106. These arousal centers can give the fundamental excitatory inputs to the thalamus 108 and the cortex 110, which can provide the arousal components of consciousness. The preoptic area (“POA”) 112 of the hypothalamus 104 is an important control center that can send inhibitory GABAergic and galanergic projections to many of the major arousal centers. NREM sleep can be initiated when the POA's 112 inhibitory network activates and thereby, blocks the arousal inputs of these centers to the thalamus 108 and the cortex 110. By activating GABAergic and galanergic projections from the POA, spindle activity (9-15 Hz) oscillations can be induced, followed shortly thereafter by slow-wave (<1 Hz) and delta (1-4 Hz) oscillations, indicative of NREM sleep.

NREM sleep is clinically divided into three stages (N1-N3) using semantic thresholds on the degree of observed delta, theta, and slow wave activity, as well as on the presence of spindles and K-complexes. Spindles are generally understood to be an intermittent rhythmic activity between the cortex 110 and the thalamus 108 that can result from the activation of GABAergic and galanergic projections, discussed above. NREM stage N1 sleep is generally defined by the presence of broadband low-frequency power. Spindle activity, in combination with slow waves and K-complexes, generally defines NREM stage N2 sleep.

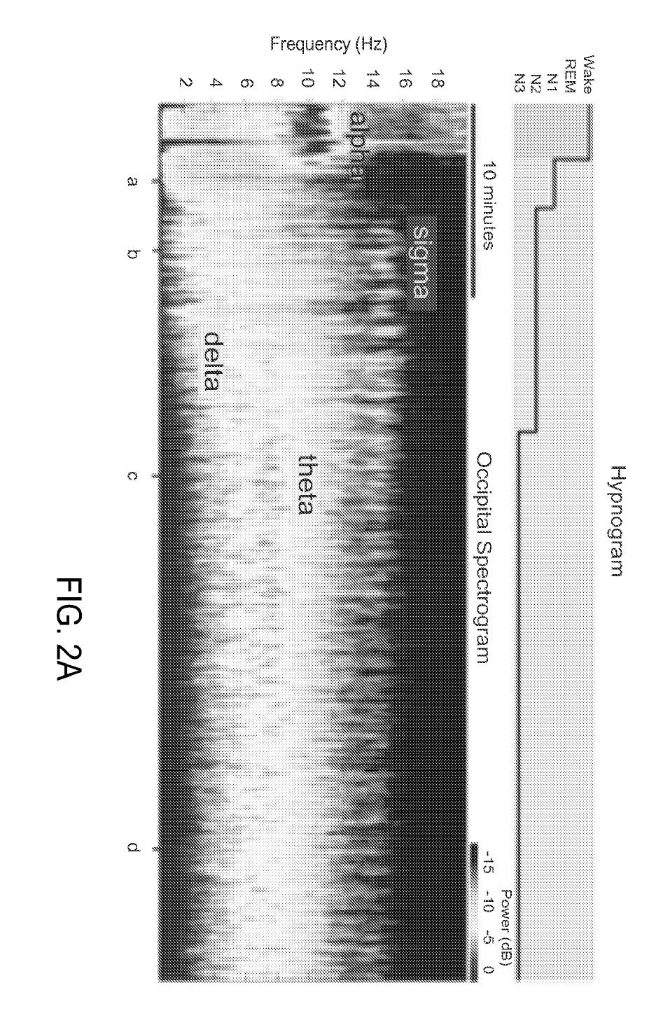

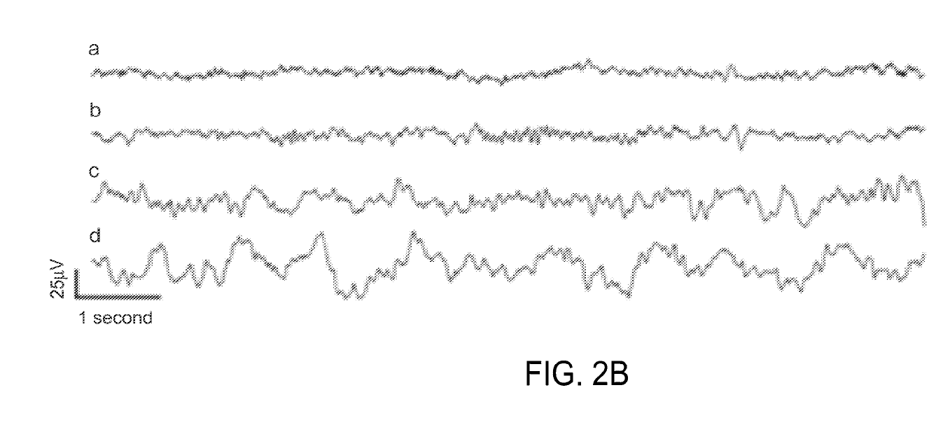

NREM stage N3, also known as slow-wave sleep, can be characterized by a predominance of slow-wave delta EEG oscillations. In one example, the slow-wave delta oscillations can result from cortical hyperpolarization, due to the decreased excitation from the arousal centers and the thalamus. FIG. 2A shows an occipital multitaper sleep spectrogram reading of a subject transitioning from a wake state to Stages N1, N2 and N3 of NREM sleep. FIG. 2B shows a time-domain measurement of the above transition.

NREM sleep can represent periods of cortical quiescence that can allow the brain to restore its energy levels. Adenosine, a product of adenosine triphosphate (“ATP”) metabolism, is known as a soporific mediator. The binding of adenosine in the basal forebrain 106 can induce POA 112 activation.

The transition from NREM sleep to REM sleep can be initiated when the cholinergic centers in the pedunculopontine tegmentum (“PPT”) 114 and the laterodorsal tegmentum (“LDT”) 116 are activated. Activity of the monoaminergic dorsal raphé and locus coeruleus (“LC”) 118 during non-REM sleep can inhibit the PPT 114 and the LDT 116 suppressing REM sleep. The dorsal raphé and the LC 118 can cease firing at REM sleep onset. Activation of the PPT 114 and the LDT 116, which project to the thalamus 108, basal forebrain 106, and to the cortex 110, which can result in the switch of the EEG from slow-wave delta oscillations to the awake-like EEG patterns which can be characteristic of REM sleep. Further, the subcoeruleus in the Pons 102 sends glutamatergic projections to spinal cord inhibitory interneurons which synapse onto the spinal alpha motor neurons. Activation of the glutamatergic pathway can produce the atonia characteristics of REM sleep.

Contrary to the REM and NREM sleep states discussed above, sedation caused by sleep medications can often be associated with beta (13-25 Hz) oscillations, which are EEG dynamics distinct from those in REM and NREM sleep states. Rather, these beta oscillations can be associated with the amnesic effects of benzodiazepines, and do not facilitate memory consolidation. Thus, the need for natural sleep can further be seen.

As stated above, activation of the LDT 116 and the PPT 114 can be critical for initiation and maintenance of REM sleep. In some embodiments, local pharmacological regimens, along with electrical stimulation, has been used to activate LDT 116 and PPT 114. Additionally, in some embodiments, REM states with atonia can be induced optogenetically by stimulating the cholinergic neurons in the PPT 114.

To initiate NREM sleep, activation of the POA 112 can be required. In some embodiments, this can be achieved for brief periods by optogenetically inhibiting orexinergic neurons in the lateral hypothalamus (“LH”) 120. Additionally, pharmaceuticals can be used to initiate an NREM sleep condition. For example high-dose administration of an anesthetic, such as those used for inducing general anesthesia or sedation, can induce EEG slow-waves. These slow-waves can differ in their characteristics depending on the specific anesthetic used. For example, propofol, a GABAergic agonist, can produce EEG slow-waves by actions at the GABAergic projections of the POA 112 onto the arousal centers of the brain. For anesthetics such as nitrous oxide and ketamine, which are NMDA antagonists, the produced slow-waves can result from the blocking of glutamatergic projections from the parabrachial (“PB”) nucleus 122 to the basal forebrain 106 and the thalamus 108.

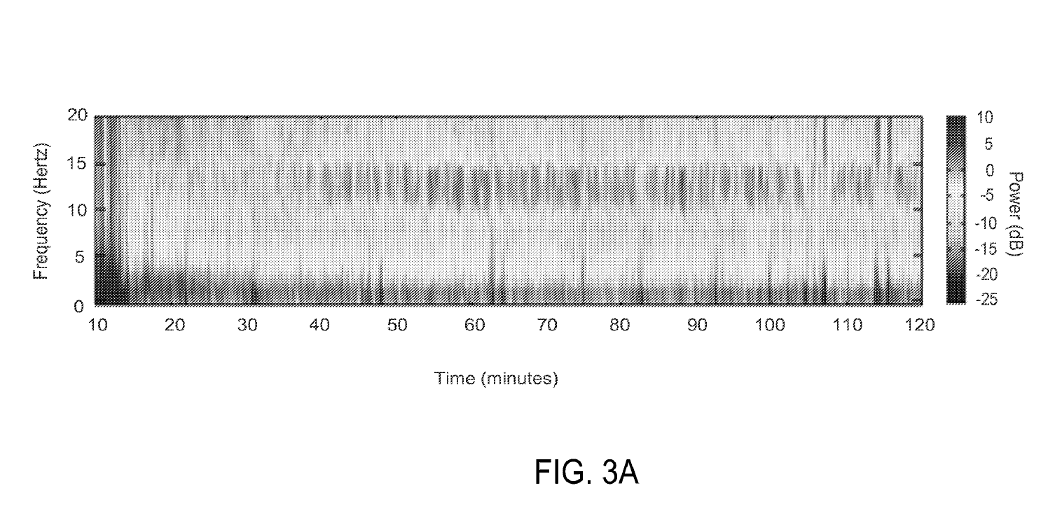

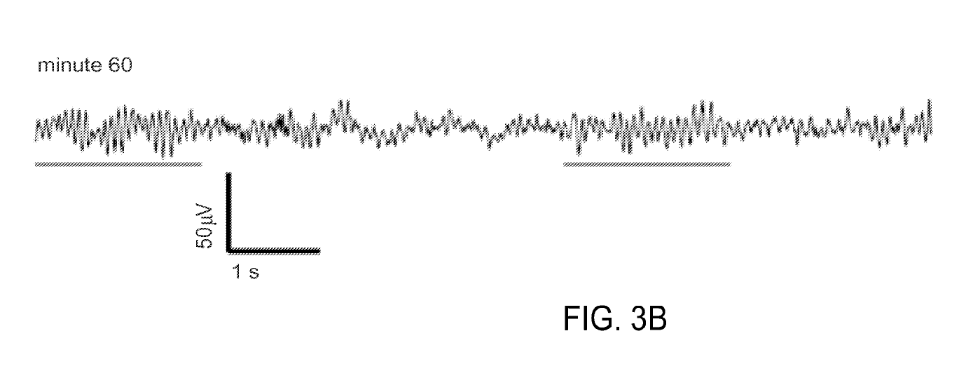

For anesthetics such as dexmedetomidine, an alpha-2 adrenergic agonist, a low-dose administration can produce spindles and slow-waves that can closely resemble stage N2 NREM sleep, as shown in FIGS. 3A-3B. In one embodiment, a low-dose of dexmedetomidine can be a range from about 0.9 mcg/kg to about 1.1 mcg/kg loading, and about 0.60 mcg/kg/hr to about 0.7 mcg/kg/hr for maintenance. The spindles and slow waves can be caused by the dexmedetomidine decreasing the release of norepinephrine from the LC 116 neurons, similar to what occurs during physiologic NREM sleep induction. This reduces the norepinephrine released into the basal forebrain 106, including the POA 112, the interlaminar nucleus of the thalamus 108, and the cortex 110. The spindles associated with low-dose administration of dexmedetomidine can result from intermittent thalamocortical communication that persists until further inhibition reduces the brain activity to only slow waves. This can produce measureable brain wave signals indicative of NREM sleep, as seen in FIGS. 3A-3B.

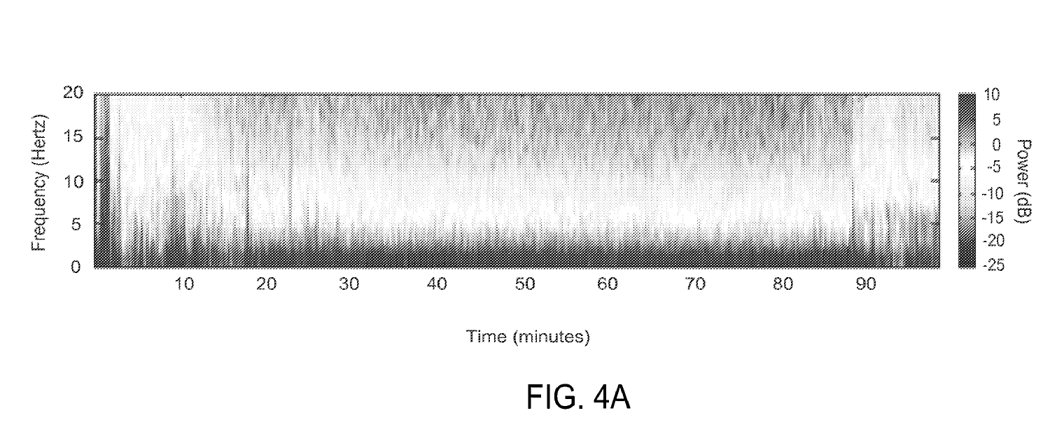

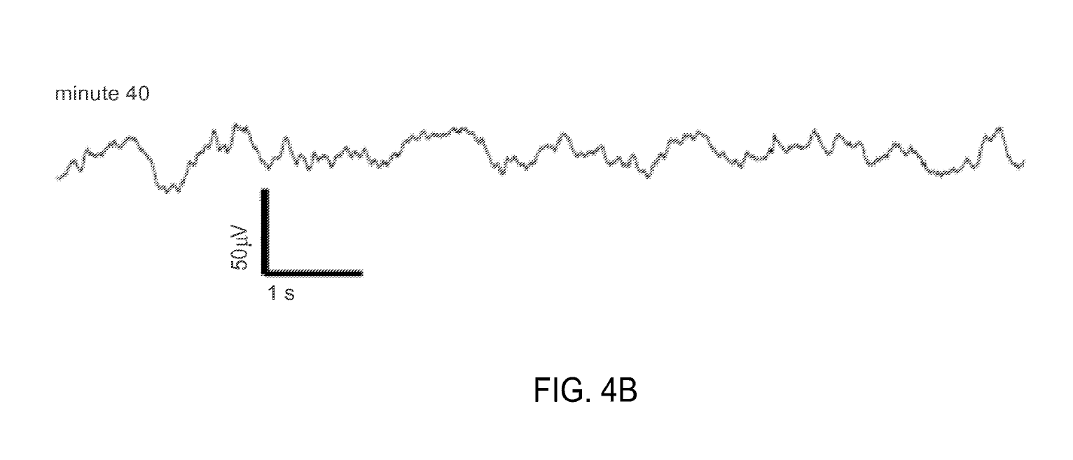

Additionally, high-doses of dexmedetomidine can result in only slow-waves being induced, as shown in FIGS. 4A-4B. In one embodiment, a high-dose of dexmedetomidine can be a range from about 0.9 mcg/kg to about 1.1 mcg/kg loading, and about 0.80 mcg/kg/hr to about 0.9 mcg/kg/hr for maintenance. The slow-waves can result from activation of the POA 112 by blocking inhibitory inputs from the LC 116 to the POA 112, and from loss of LC 116 excitatory adrenergic inputs to the basal forebrain 106, intralaminar nucleus of the thalamus 108, and to the cortex 110, via the further reduction of norepinephrine released into the basal forebrain 106. Thus, reduced arousal and slow-waves can be created by activation of the POA 112, activation of the thalamic reticular nucleus (TRN) 124, or by inactivation of the LC 116, PB 122, LH 120, ventral tegmental area (VTA) 126 or, in some embodiments, one or more of the other arousal centers.

Similarly, analogs of dexmedetomidine—compounds known to act as alpha-2 adrenergic receptors—can further be used with similar results to those seen when using dexmedetomidine. These can include clonidine, guanfacine, xylazine, and medetomidine. The above examples can all be used to induce NREM sleep.

Based on the above, it can be possible to control specific brain circuits using various means to produce decreased arousal that can result in cycling between NREM and REM states in a manner that can mimic natural sleep. This control is herein referred to as biomimetic sleep. Biomimetic sleep can be achieved, in one embodiment, by using pharmacological means. Alternatively, optogenetic and/or electrical stimulus can be used as well, either alone or in combination with pharmaceuticals.

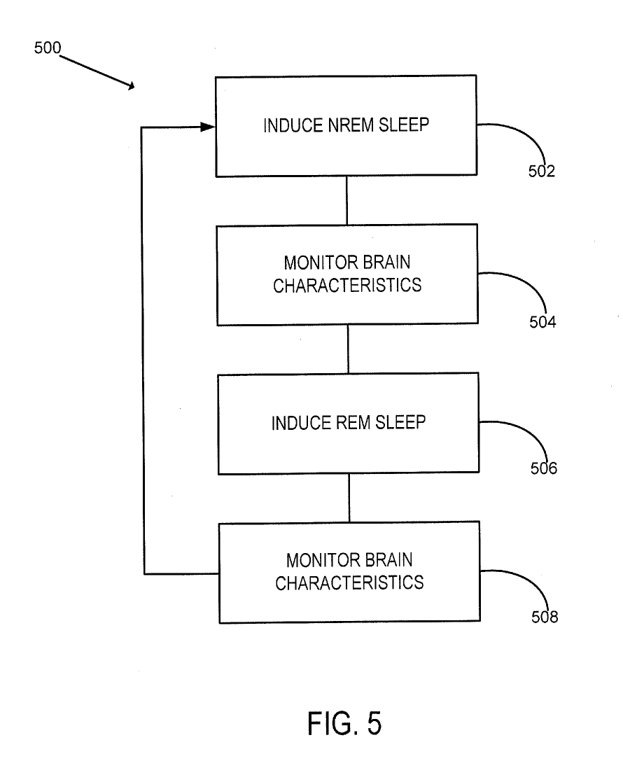

FIG. 5 shows a process for inducing natural sleep 500, illustrating the steps of producing natural sleep in a subject. At step 502, NREM sleep can be induced. As stated above, NREM sleep is generally characterized by slow-wave oscillations in the brain. To induce slow-wave oscillations, several approaches can be employed. In one embodiment, optogenetic and/or electric stimulation can be used to activate GABAergic neurons in the POA 112 or the TRN 124. Optogenetic and/or electric stimulation could also be used to inactivate glutamatergic neurons in the PB 122 nucleus. Alternatively, optogenetic and/or electric stimulation can be used to inactivate noradrenergic neurons in the LC 116. Additionally, optogenetic and/or electric stimulation can be used inactivate the dopaminergic neurons in the VTA 126. The above approaches can induce a significant decrease in excitatory input to the cortex from the brainstem and the thalamus 108. Of the four approaches discussed above, activation of the GABAergic projection in the POA 112 could most closely mimic the manner in which NREM sleep is naturally initiated. In one example, the median preoptic (“MPO”) nucleus and the ventrolateral preoptic (“VLPO”) nucleus, both located within the PB 122 nucleus, can be targeted. Both the MPO and the VLPO have been known to be implicated in the initiation of NREM sleep.

Finally, pharmaceuticals can be used to induce NREM sleep. In one example, dexmedetomidine can be used to induce NREM sleep, as discussed above. Additionally, NREM sleep can be induced using Designer Receptors Exclusively Activated by Designer Drugs (“DREADDS”) along with adeno associated virus vectors to activate the POA 112 or the TRN 124, or, alternatively, to inactivate the LC 116. The DREADDS induced NREM sleep can last 3-8 hours.

At process block 504, the subject can be monitored to determine the effects of the NREM inducement methods discussed above. In one embodiment EEG measurements can be taken. Alternatively, electromyography (“EMG”) techniques and/or electroculogram (“EOG”) can be used to monitor the induced NREM sleep. In addition, autonomic and systemic physiological variables such as respiration, heart rate, heart rate variability, blood pressure, and galvanic skin response (“GSR”) can also be used to monitor NREM sleep. Respiration could be measured with either plethysmography or capnography, or some combination thereof. Heart rate and heart rate variability could be estimated from electrocardiogram or pulse-oximetry, in one example. Blood pressure can be measured, in one embodiment, using a non-invasive blood pressure cuff. Moreover, combinations of these measurements—EEG, EMG, EOG, respiration, heart rate, heart rate variability, blood pressure, and GSR—could also be used to monitor NREM sleep. The subject can be monitored to determine the extent to which the brain states created by the administered pharmaceuticals and/or the optogenetic or electrical manipulation of the brain, approximate an NREM sleep state. Additionally, the progressive increase in POA 112 activation and/or inactivation of other brain sites can be monitored to determine the extent of deeper NREM states.

At process block 506, a REM sleep state can be induced. As discussed above, activation of the PPT 114 and LDT 116 can be critical for induction of REM sleep. In one embodiment, optogenetic and/or electrical stimulus can be used to activate the PPT and LDT to induce REM sleep. Alternatively, the PPT 114 and LDT 116 can be stimulated using pharmacological means, such as with cholinergic drugs. For example, administration of a cholinergic agonist, such as rivastigmine, could be used to induce REM sleep. Rivastigmine can be administered either orally or intravenously.

Generally, REM sleep can only be initiated once a subject is in NREM sleep. Thus, there are several methods for inducing REM sleep using the above described methodologies. In one embodiment, the subject can be allowed to sleep naturally. Alternatively, the NREM sleep can be induced using dexmedetomidine, or other similar alpha-2 adrenergic mechanisms. DREADDS can further be used to induce the NREM sleep, as discussed above.

The subject's brain waves can then be monitored to determine when the subject enters NREM sleep. The subject's brain waves can be measured using EEG, EMG, or a combination thereof. Additionally, other brain wave monitoring devices could be used to monitor the subject's brain waves. Once the subject is in NREM sleep, the cholinergic circuits in the PPT 114 circuit can be activated. The cholinergic circuits in the PPT 114 circuit can be activated, in one embodiment, using optogenetic stimulation. Alternatively, the cholinergic circuits in the PPT 114 can be activated using electrical stimulus or pharmacological regimens. In one example, the administration of intravenous rivastigmine can be used to achieve cholinergic activation. In one embodiment, a combination of optogenetic stimulus, electronic stimulus, and/or pharmacological regimens can be used to activate the cholinergic circuits in the PPT 114. This activation can be repeated on the LDT 116 using the above methodologies. Additionally, in some embodiments, certain pharmaceuticals known to act as cholinergic receptors can be used to promote REM sleep states. For example, donepezil, or rivastigmine can be administered at particular points in time during the biomimetic sleep process to promote REM sleep.

In a further embodiment, optogenetic and/or electrical stimulation can be used to induce both NREM and REM sleep states. This can require specific targeting of two separate sites within the brain. For optogenetic, implementation, activation of two distinct neuronal cell types can require implanting optogenetic devices in two distinct area. These distinct areas can be monitored and recorded using EEG and/or EMG while stimulating the relevant brain regions.

At process block 508, a subject can be monitored to determine the effects of the REM inducement methods discussed above. Monitoring can be performed using EEG, EMG, EOG, respiration, heart rate, heart rate variability, blood pressure, and GSR, or some combination thereof. The subject can be monitored to determine the extent to which the brain states created by the administered pharmaceuticals and/or the optogenetic or electrical stimulation of the brain approximate an REM sleep state. Additionally, the activity within LDT 116 and PPT 114 and/or inactivation of other brain sites can be monitored to determine the REM sleep state.

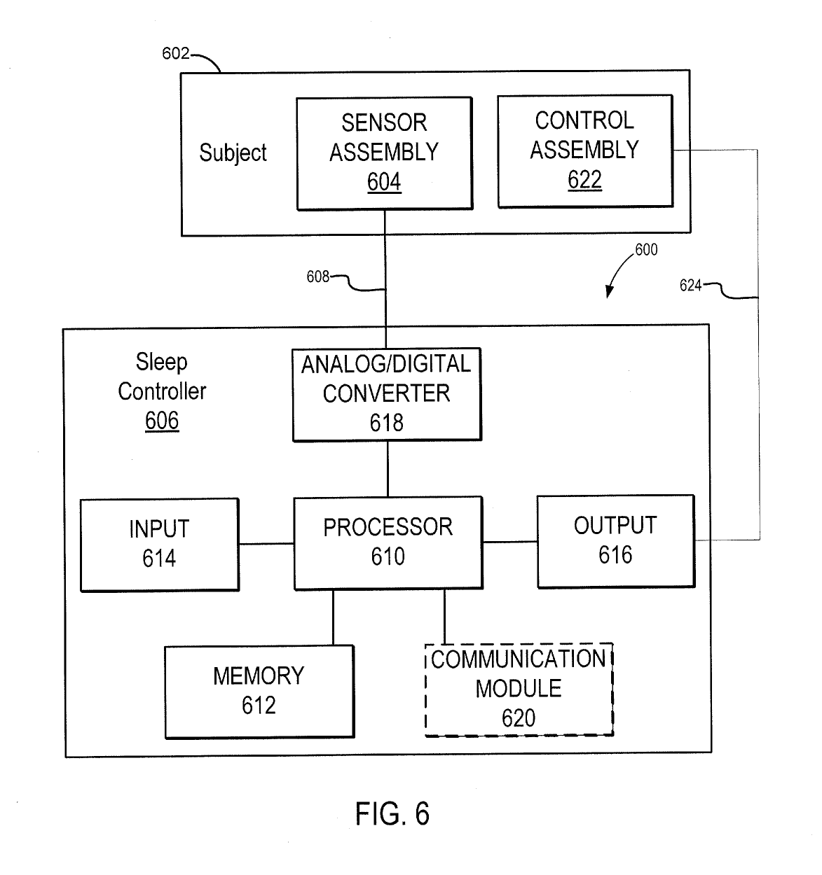

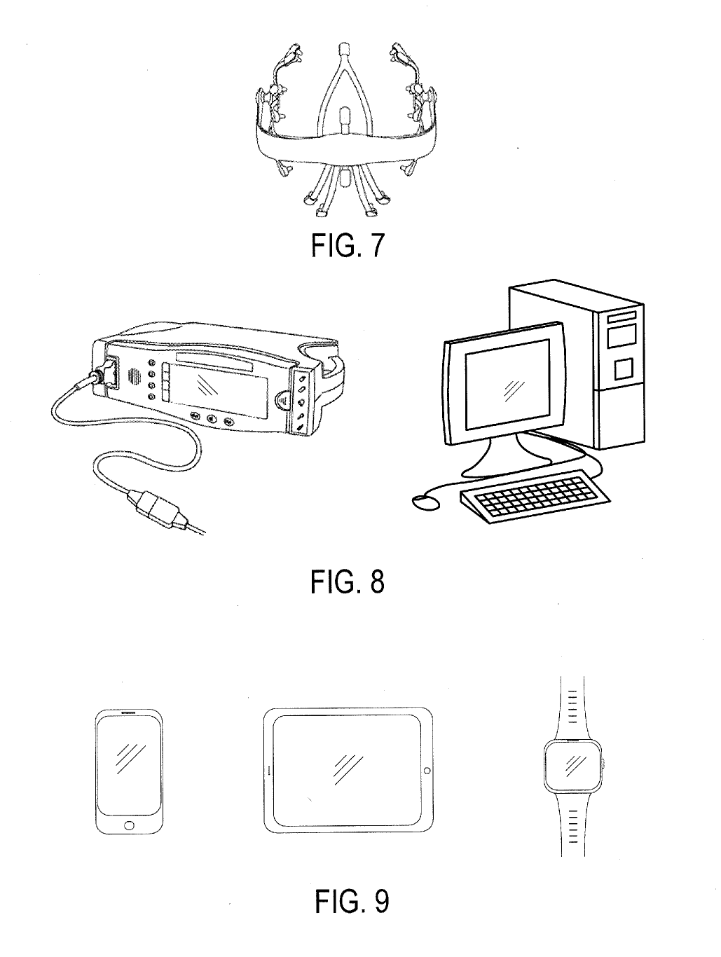

Turning now to FIG. 6, an example brain-machine interface (“BMI”) 600 for controlling the sleep of a subject 602 can be seen. The BMI 600 can have a sensor assembly 604 for monitoring a subjects brain waves. In one embodiment, the sensor assembly 604 can be an EEG type sensing assembly. Alternatively, the sensor assembly 604 can be an EMG type sensing assembly. The sensor assembly 604 could also include both EEG and EMG technology for monitoring brain waves. Other brain wave measuring instruments as known in the art could also be incorporated into the sensor assembly 604. In one example, the sensor assembly 604 can include a plurality of sensors for monitoring brain waves and/or other characteristics from various scalp positions, as shown in FIG. 7. In another example, additional sensors, possibly positioned on other parts of the body, can be used to measure EMG, respiration, heart rate, heart rate variability, blood pressure, and GSR, which, in turn, could be used to help infer brain states.

The BMI 600 can also have a sleep controller 606. The sleep controller 606 can communicate with the sensor assembly 604 via a wired or wireless communication link 608. In one embodiment, the communication link 608 can be a serial data interface. Alternatively, the communication link 608 can be a universal serial bus (“USB”), fire wire, Ethernet, fiber optic, or other data communication protocol as known in the art. Further, the communication link 604 can be a wireless communication link, such as Wi-Fi, Bluetooth,® cellular (3G, 4G, LTE), or other wireless communication protocol, as non-limiting examples. The communication link 608 can further be a plurality of parallel analog data channels.

The sleep controller 606 can further have a processor 610. In one non-limiting example, the processor 610 can be a standard processor such as an Intel® based processor. Alternatively, other non-limiting examples of the processor 610 can be a dedicated processor such as an application specific integrated circuit (“ASIC”), a field-programmable gate array (“FPGA”) and/or a graphical processing unit (“GPU”). The processor 610 can be in communication with a memory device 612, an input module 614 and an output module 616. The processor 610 can further be configured to carry out any number of steps for operating the BMI 600. In addition, the processor 600 may be programmed to pre-process data obtained from the subject 602 using instructions stored in the memory 612. For example, the processor 610 can be configured to perform signal conditioning or pre-processing, such as scaling, amplifying, or selecting desirable signals, or filtering interfering or undesirable signals. In addition, the processor 610 can be configured to generate EEG sleep data using, for example, a scalp Laplacian montage, and/or perform a source localization analysis.

The processor 610 can be configured to analyze and identify signatures associated with data obtained from the subject 602 in order to characterize sleep dynamics and the onset of sleep. Specifically, the processor 610 may be configured to carry out sleep analysis using spectral analysis, autoregressive time series modeling, coherence analysis, global coherence analysis, and so forth. In some aspects, the processor 610 may assemble one or more spectrograms using a multitaper technique. Furthermore, the processor 610 may be configured to analyze specific events, features, time-scales, and frequency components of the data associated with different diagnostic features.

In some embodiments, the processor 610 can utilize one or more generated spectrograms to identify specific signatures or signal features associated with particular stages of sleep. For instance, the processor 610 can utilize generated spectrograms with parameters optimized to identify arousals, spindles, K-complexes, and other features, or with parameters optimized to identify ultradian features. The processor 610 can also be configured to perform automatic detection of subject-specific stationary slow, alpha, and spindle peak oscillation frequencies. The processor 610 can also track, over time, subject-specific non-stationary frequency peaks including sigma, alpha, theta, and slow, and other signature oscillations. The processor 610 can also compute changes in the time constant in spectral power bands between different stages of sleep. The processor 610 can further utilize a multinomial model of sleep to estimate from EEG data one or more probability and uncertainty of a subject 602 being in a given state of sleep at a given point in time. The processor 610 may also be configured to normalize spectral band power using data obtained from multiple subjects by using percentile-based normalization functions, including percentile-based indicator functions. In some aspects, identified signatures or signal features may be utilized to determine a sleep condition of a subject 602, or an effectiveness of an administered pharmacological agent on the sleep on of the subject 602. The processor 610 can further be configured to determine a sleep fragmentation using computed spectrograms.

The processor 610 may be further configured to generate and provide a report either intermittently, or in real time, via output 616, which may include a display. The report can be in any form (i.e. textual, graphical, etc.), and include any information, including information related to acquired and processed physiological data, such as time-series waveforms or traces, time-frequency representations, power spectra, multitaper spectrograms, and so on. On some aspects, the report may include an indication or index related to the degree to which the subject 602 is awake at one or more points in time. Also, the report may include descriptions regarding a state of wakefulness or sleep of the subject 602. In other embodiments, the report may characterize a sleep or sleep onset process. For instance, the report may include estimated probabilities, as well as confidence intervals thereof. The report may also include information regarding an identified sleep condition, effectiveness of an administered pharmacological agent, etc. The report may also include information derived from a comparison between subject 602 data and data obtained from a population.

The processor 610 can further be configured to execute a feedback based (i.e. closed loop) control algorithm. In one example, the processor 610 can execute a proportional-integral-derivative (“PID”) feedback control algorithm. In further embodiment, the processor 610 can execute a stochastic feedback control algorithm. Other closed loop control algorithms could also be used, as applicable. In one embodiment, the processor 610 can use the data from the sensor assembly 604 as closed-loop feedback data. In some embodiments, subject 602 characteristics such as age, weight, height, and gender, for example, can be used to establish pharmacokinetic and pharmacodynamics models that can be used within the control algorithm. In other embodiments, individualized patient response characteristics, including pharmacokinetics and pharmacodynamics, could also be estimated from initial drug administration data as part of the control algoritm.

In embodiments where the sensor assembly 604 provides an analog signal to the sleep controller 606 via the communication link 608, the sleep controller 606 can further have an analog/digital converter (“ADC”) 618. The ADC 618 can convert analog signals provided by the sensor assembly 604 into digital signals compatible with the processor 610. The processor 610 can also optionally be connected to a communication module 620. The communication module 620 can output data to an external device (not shown). In one embodiment, the communication module 620 can output data gathered by the sleep controller 606 to a remote device (not shown), such as a personal computer, a smart phone, a tablet device, or other personal electronic device. In one embodiment, the communication module 620 can transmit data to the remote device using a wireless communication protocol. Non-limiting examples of the wireless communication protocol can be Wi-Fi (IEEE 800.11x), Bluetooth, cellular (3G, 4G, LTE), near field communication (NFC), or other wireless communication protocol as known in the art. In one embodiment, the remote device can be a cloud-based server, which can allow for a user to access the data from any remote location with access to the cloud server.

The sleep controller 606 can output data via the output module 616 which can be sent to a control assembly 622 via an output data line 624. The control assembly 622 can be coupled to the subject 602 and used to provide stimuli necessary for the induction and maintenance of both NREM and REM sleep, as will be discussed in more detail below. In one embodiment, the control assembly 622 can be a pharmaceutical delivery device, capable of administering one or more pharmaceuticals to the subject 602. The control assembly 622 can regulate the doses of the pharmaceuticals as required to maintain a sleep state, or to transition to a different sleep state. In one example, the control assembly 622 can administer and regulate doses of dexmedetomidine. In a further embodiment, the control assembly 622 can administer various other pharmaceuticals to induce and maintain different sleep states. In one non-limiting example, the control assembly 622 can administer a first pharmaceutical to induce and maintain a first sleep state, such as NREM. The control assembly 622 can then administer a second pharmaceutical to induce and maintain a second sleep state, such as REM. In further embodiments, the control assembly 622 can be configured to administer more than two different pharmaceuticals, as needed.

In another embodiment, the control assembly 622 can be used to maintain and/or transition between sleep states by controlling a subject using optogenetic implants. The optogenetic implants can be placed in relevant portions of the brain for regulating sleep. The control assembly 622 can be coupled to the subject 602, and in electronic communication with the optogenetic implants. In one embodiment, the control assembly 622 can activate the optogenetic implants via a radio frequency (RF) signal. Alternatively, the control assembly 622 can use inductive coupling to activate the optogenetic implants. In some embodiments, the control assembly 622 can activate the optogenetic implants using physical leads connected to the implants. Further, the control assembly 622 can activate the optogenetic implants using means known in the art.

The BMI 600 can operate either independently, or in collaboration with any computer, system, device, machine, mainframe, database, server or network, as shown in the examples of FIG. 8. In some aspects, the BMI 600 may be a portable or wearable device or apparatus, such as the shown in FIG. 9. Alternatively, the BMI 600 can be configured to communicate with such portable devices, for example, via the communication module 620.

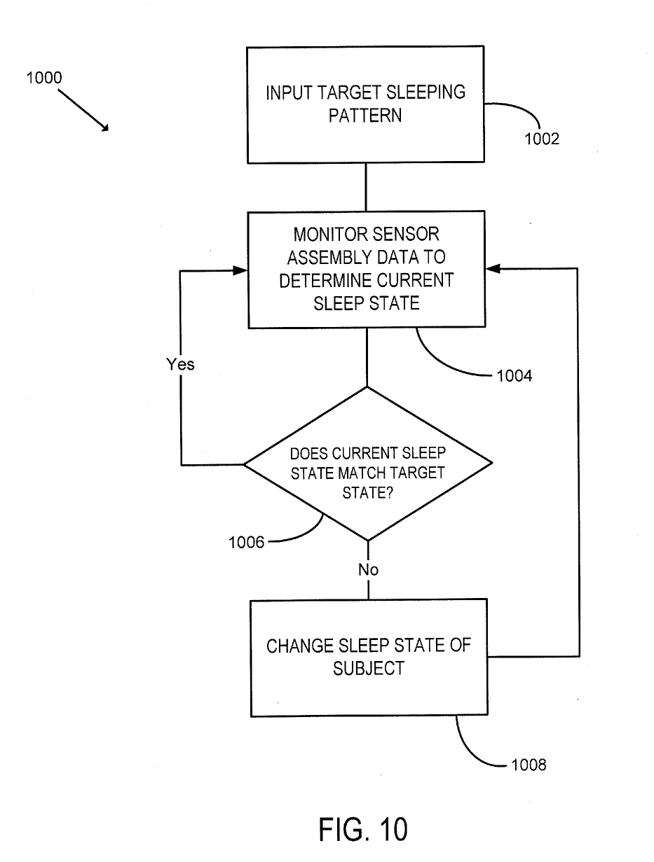

To induce and maintain biomimetic sleep, the BMI 600 can use a closed loop control systems. FIG. 10 provides a control process 1000 of controlling and maintaining biomimetic sleep with the BMI 600. At process block 1002, a user can input a desired sleep pattern for the subject 602 via the input module 614. For example, two hours of NREM-REM cycling with four, twenty-five minute periods of NREM sleep and five minutes of REM sleep after each twenty-five minute period of NREM sleep. In another non-limiting example, the NREM sleep state and the REM sleep state can transition every ninety minutes. Other durations and cycling intervals can also be input at process block 1002, as desired. The NREM sleep and REM sleep cycles can be set as target states for the subject's sleep state. These target states can be used as set point values for a closed-loop control algorithm executed by the processor. In one embodiment, the feedback control algorithm can be a PID control algorithm. Alternatively, the feedback control algorithm can be a stochastic control algorithm. The processor 610, executing a closed-loop control algorithm, can constantly maintain the desired sleep state based on the data received from the sensor assembly 604.

At process block 1004, the sleep state of the subject 602 is determined. In one embodiment, the processor can determine the sleep state by observing the brain wave data provided by the sensor assembly 604, and evaluating the brain waves to establish if the subject 602 is in NREM or REM sleep, as discussed above. For example, if the brain wave data provided by the sensor assembly 604 shows the presence of broadband low-frequency power only, the processor 610 may determine that the subject is in NREM stage N1 sleep. If the brain wave data provided by the sensor assembly 604 shows spindle activity, in combination with slow waves and K-complexes, the processor 610 may determine that the subject is in NREM stage N2 sleep. If the brain wave data provided by the sensor assembly 604 shows slow-wave delta EEG oscillations, the processor 610 may determine that the subject is in NREM stage N3 sleep. Additionally, if the brain wave data provided by the sensor assembly 604 shows EEG and/or EMG patterns indicative or REM, such as lower voltage, high-frequency activity, intermittent burst of alpha waves, arousal patterns, or increased EMG and EOG activity. The processor 610 may determine that the subject is in REM sleep. In one example, awake patterns can be seen in the form of increased desynchrony, and low-amplitude, irregular EEG activity with an absence of slow-delta and spindle activity. The above examples are non-limiting, and other data can also be evaluated separately or in combination with the above examples to determine the sleep state; for example, heart rate, brain activity, muscle activity, respiration activity, cardiac activity, eye movement, galvanic skin response, blood oxygenation, as well as motion, pressure, temperature, force, sound, flow, etc., can all be indicators of sleep states.

The control process 1000 can then evaluate if the determined sleep state of the subject 602 matches the desired target sleep state at process block 1006. If the determined sleep state is the target sleep state, the control process 1000 returns to process block 1004 to continue to determine the sleep state of the subject 602. The control process can continue to determine the sleep state of the subject 602 until the duration of the induced biomimetic sleep input at process block 1002 is completed. Alternatively, the control process 500 can stop determining the sleep state of the subject 602 if it is determined that the subject 602 is no longer in a sleep state. The control process 1000 can also stop determining the sleep state of the subject where the subject awakens and stops the control process 1000.

Where the determined sleep state does not match the target sleep state, the control process 1000 can change the sleep state of the subject 602 at process block 1008. To change the sleep state of the subject 602, the processor 610 can output a signal via the output module 616 to the control assembly 622 indicating that the current sleep state should be changed. The control assembly 622, can, based on the signal received via the output module 616, can then effect a change in the subject's 602 sleep state. In one example, if the target sleep state is REM, and the current sleep state is NREM, the control assembly can increase PB 122 stimulation (glutamatergic inactivation) to enhance the NREM state, and then stimulate the cholinergic pathways, such as those in the PPT 114, to induce the transition to the REM sleep state. Alternatively, if the target sleep state is NREM and the actual state is REM, the controller can turn off the cholinergic activation in order to allow the transition to the NREM state. As stated above, the control assembly 622 can induce NREM and REM sleep state transitions using various methods, including optogenetic stimulation, electronic stimulation, and/or pharmacological regimens. Further, the control assembly 622 can be used in conjunction with a closed-loop control system to allow for the sleep state of a subject 602 to be constantly regulated and maintained.

### EXAMPLES

Optogenetic devices were implanted in two distinct area of a subject brain to allow for optogenetic activation of two distinct neuronal cell types. EEG and EMG activity was recorded and monitored while stimulating the distinct brain regions. The different cell types of the different brain sites were activated using a transgenic mouse for one area and a virus containing the specific promoter or an electrical stimulation was used to active the other brain sites. For example, a first distinct area could be the PB 122 glutamatergic cells, which could be activated using a vesicular glutamate transporter promoter in a virus or combined with an optogenetic stimulation or an electrical stimulation to activate the cholinergic neurons in the PPT 114. A multisite stimulating array is then used to inactivate glutamatergic cells to induce an NREM sleep state. Once the NREM sleep state has been verified as adequate, the PPT 114 can be activated using optogenetic or electrical stimulation to induce the REM sleep state.

In another example, biomimetic sleep could be initiated with the drug dexmedetomidine, employing a dose that could range between 0.5 to 1.5 mg/kg over a period of five to ten minutes. The electroencephalogram (EEG) could be used to monitor the patient's brain state and guide administration of the drug, and could also be used as a control signal for feedback control of biomimetic sleep. The dose could be adjusted to account for individual subject's responses, and to achieve different sleep brain state trajectories, depending, for instance, on the therapeutic goals. Besides dexmedetomidine, a number of other compounds could be used in any particular implementation of biomimetic sleep. For instance, analogs of dexmedetomidine known to act as alpha-2 adrenergic receptors, such as clonidine, guanfacine, xylazine, or medetomidine could be used to promote biomimetic non-rapid eye movement sleep states. In addition, drugs known to act as acetylcholinergic receptors, such as donepezil or rivastigmine, could be used to promote biomimetic rapid eye movement sleep states.

As may be appreciated, the provided system and method may be implemented in a variety of systems and devices. For instance, some implementations can includes systems and devices for research or commercial laboratory sleep monitoring, for home sleep monitoring, as well as a number of commercial products, such as self-improvement or fitness applications, including wearable consumer products or mobile devices.

The present invention has been described in terms of one or more preferred embodiments, and it should be appreciated that many equivalents, alternatives, variations, and modifications, aside from those expressly stated, are possible and within the scope of the invention.

1. A method for inducing natural sleep in a subject, the method comprising:
inducing a first sleep state;
monitoring a first plurality of characteristics of a brain of the subject to verify the subject is in the first sleep state;
inducing a second sleep state after the subject has been in the first sleep state for a pre-determined amount of time; and
monitoring a second plurality of characteristics of the brain of the subject to verify the subject is in the second sleep state.

2. The method of claim 1, wherein the first sleep state is a non-REM sleep state, and the second sleep state is a REM sleep state.

3. The method of claim 1, further comprising the step of transitioning between the first sleep state and the second sleep state for a pre-determined number of transitions.

4. The method of claim 1, wherein the monitoring of the first plurality of characteristics and the monitoring of the second plurality of characteristics is performed using an electroencephalogram.

5. The method of claim 1, wherein the pre-determined amount of time is 90 minutes.

6. The method of claim 1, wherein the first sleep state is induced using a pharmaceutical.

7. The method of claim 6, wherein the pharmaceutical is dexmedetomidine.

8. The method of claim 5, wherein the pharmaceutical is Designer Receptors Exclusively Activated by Designer Drugs.

9. The method of claim 1, wherein the second sleep state is induced using at least one stimuli.

10. The method of claim 9, wherein the at least one stimuli is an optogenetic stimuli.

11. The method of claim 10, wherein the optogenetic stimuli is performed by optogenetic implants implanted in the brain of the subject.

12. The method of claim 9, wherein the at least one stimuli is an external electrical stimuli.

13. A system for controlling sleep states of a subject, the system comprising:
a sensor assembly, wherein the sensor assembly is capable of measuring brain activity;
a sleep controller, the sleep controller in communication with the sensor assembly and including an input, an output and a processor, wherein the sleep controller analyzes the measured brain activity measured; and
an external control module, the external control module in electronic communication with the sleep controller and capable of inducing a sleep state in a subject.

14. The system of claim 13, wherein the external control module can induce at least one of a not rapid eye movement sleep state and a rapid eye movement sleep state.

15. The system of claim 14, wherein the external control module can induce the not rapid eye movement sleep state by administering a pharmaceutical.

16. The system of claim 15, wherein the pharmaceutical is dexmedetomidine.

17. The system of claim 14, wherein the external control module can induce the rapid eye movement sleep state by transmitting a stimuli to the brain of the subject.

18. The system of claim 17, wherein the stimuli is an electrical stimuli.

19. A method for inducing biomimetic sleep in a subject, the method comprising:
inducing a first sleep state;
monitoring a first plurality of characteristics of a brain of the subject to verify the subject is in the first sleep state;
inducing a second sleep state after the subject has been in the first sleep state for a first pre-determined amount of time;
monitoring a second plurality of characteristics of the brain of the subject to verify the subject is in the second sleep state; and
transitioning between the first sleep state and the second sleep state for a pre- determined number of transitions, wherein the subject is in the second sleep state for a second pre-determined amount of time.

20. The method of claim 19, wherein the first sleep state is a non-REM sleep state, and the second sleep state is a REM sleep state.
